(19) United States
(12) Patent Application Publication
(10) Pub. No.: US 2005/0049207 A1
(43) Pub. Date: Mar. 3, 2005

(54) Method of treating and preventing cancer

(76) Inventor: Doug A. Kaufmann, Rockwall, TX (US)

Correspondence Address: Michael A. O'Neil;Michael A. O'Neil, P.C., Suite 820, 5949 Sherry Lane, Dallas, TX, 75225, US

(21) Appl. No.: 10/674,145

(22) Filed: Sep. 29, 2003

Related U.S. Application Data
(60) Provisional application No. 60/499,976, filed on Sep. 3, 2003.

(51) Int. Cl. A61K 31/7048; A61K 31/496; A61K 31/4196; A61K 31/4178

(52) U.S. Cl. 514/28; 514/254.07; 514/383; 514/397

(57) ABSTRACT

This invention comprises a method of treating cancer. Specifically, the method involves administering one or more anti-fungal agents in amounts, at frequencies, and for durations which are effective in preventing and treating cancer. The method further comprises the administration of a low carbohydrate diet which may be used either in combination with the aforesaid anti-fungal agent or separately therefrom.

### CLAIM OF PRIORITY

This application claims priority based on provisional patent application Ser. No. 60/499,976 filed Sep. 3, 2003.

### TECHNICAL FIELD

This invention relates generally to the prevention and treatment of cancer, and more particularly to the prevention and treatment of cancer utilizing anti-fungal agents and/or anti-fungal dietary regimes.

### BACKGROUND AND SUMMARY OF THE INVENTION

As used herein the term “cancer” means benign neoplasms, malignant neoplasms, cancers or growths in the bones and soft tissues—including skin,—of the body; cancers of the central nervous system, and cancers of the organs and various glands, including the reproductive glands, in the body. The term also encompasses cancers and conditions in the bone marrow and bone marrow cells—conditions such as myelodysplasia, multiple myeloma and other monoclonal gammopathies and plasma cell dyscrasias, and other bone marrow malignancies. In addition, it encompasses malignancies of the blood cells, lymphatics and lymph nodes (lymphoreticular system), such as leukemia—both acute and chronic forms—and lymphoma (Hodgkin's and non-Hodgkin's variations). The term “cancer” also encompasses cancers related to AIDS.

Advances in cancer treatment techniques, including surgery, chemotherapy, radiation, etc. are well publicized. Unfortunately, none of these techniques is useful until the existence of cancer has been confirmed utilizing laboratory techniques which often does not occur until the cancer has become life threatening. Other than the nationwide campaign to eliminate tobacco usage, no procedure exists for preventing cancer from occurring. Equally unfortunate is the fact that no technique currently exists for treating cancer-like symptoms prior to confirmation that cancer does in fact exist.

The present invention comprises the use of antifungals, both medicinal and naturally-occurring, in the prevention and treatment of cancer in mammals. The use of the antifungal substances pertains to preventive use, empiric use, and specifically-directed use of the antifungals toward the treatment and prevention of cancer in mammals. Preventive use means using antifungal substances to avoid instances of cancer altogether. Empiric use indicates the use of an antifungal substance when a cancer is suspected due to cancer indicating symptoms. Specifically-directed use applies when a cancer has been confirmed by laboratory tests such as tissue biopsies, tumor markers, and other conventional or alternative methods of testing for cancer.

The present invention also comprises the use of diet for both the treatment and the prevention of cancer. A specific diet is used in conjunction with or apart from antifungal medications or naturally-occurring antifungal substances. The diet is a low-carbohydrate type of diet that is designed to be low in naturally-occurring carcinogenic and immune-suppressing fungal toxins, known as mycotoxins.

The present invention further comprises the use of both the diet and antifungal substances either as the sole therapies for the cancer or in combination with either conventional chemotherapy, surgery, radiation, immune-modulating therapies, thermal-related therapies, cancer vaccines, or any combination of conventional therapies.

In addition, the present invention comprises the use of the specific diet, with or without antifungal substances, if both or either are used in conjunction with any alternative type of cancer therapy. Alternative therapies are those that are currently defined by the National Institutes of Health's Office of Alternative Medicine and/or the National Center for Complimentary and Alternative Medicine. Such alternative practices may include nutrition, massage, chiropractic manipulation, mind-body medicine, Ayurveda, naturopathy, homeopathy, reflexology, magnet therapies, hypnosis, vitamin and herbal therapies, biofeedback, osteopathic manipulation therapy, aromatherapy, and others.

### DETAILED DESCRIPTION

The Prevalence of Cancer and the Need to Acknowledge the True Cause of Cancer

Reuters. Jan. 3 2002. Rate of Cancer Highest in North America: Study. (Pisani, Paola, et al. Estimates of the World-wide prevalence of cancer for 25 sites in the adult population. International Journal of Cancer 2002;97:72-81).

Despite our advances in technology, cancer in America seems to more popular than in other parts of the world. One factor may be that we don't screen for as many of the carcinogenic mycotoxins in our food supply as some other countries do, and that we still allow contaminated grains to go to livestock that ends up on our tables anyhow. In addition, our easy access to antibiotics—clear risk factors for fungal infections—may be proving to be more deleterious in the long run than helpful in the short term.
News Briefs: Report to the Nation: Cancer death rates drop, but total number of cases rises. CA-A cancer journal for clinicians. July/August 2002. Vol. 52, No. 4.

Cancer death rates fell by about one percent per year between 1993 and 1999. But this year's report stated that the actual numbers of cancer cases and deaths were up because the age-standardized incidence rate (expressed in new cases per 100,000 people per year) is holding steady; yet the US population is growing larger and many more people are living into age ranges where cancer is more common. If these population trends continue and incidence rate remain the same, by the year 2050 there could be twice as many people diagnosed with cancer each year.
Begley, S. New Statistics show and increase in cancer rates. Wall Street Journal, Oct. 16, 2002.

Previous indications of a decline reflected significant delays in reporting cancer cases. More accurate information about cancer rates presents a grimmer picture. The reanalysis shows that breast cancer rates actually have been rising 0.6% a year since 1987. Also, the reanalysis on lung cancer in women shows it has been rising 1.2% a year since 1996. Melanoma has actually been soaring at 4.1% a year since 1981. Prostate cancer has actually been rising 2.2% a year since 1995. Colorectal cancer cases are 3% higher than originally reported.

The Downside of Radiation Therapy, and Mammography, and Chemotherapy

Mason, R. et al. Radiation-induced sarcoma of the breast. JAOA. Vol 96 No 6. June 1996.

A small but growing number of radiation-induced sarcomas after breast-conserving surgery for carcinoma have been reported.

Radiation-induced carcinogenesis is a well-recognized phenomenon. Since the first report by Frieben in 1902, which described a case of squamous cell carcinoma on the hand of an x-ray technician, numerous reports have followed. Of particular interest, and with reference to this case, is the number of radiation-induced sarcomas that have been reported in the literature after treatment for breast carcinoma.

Radiation-induced sarcomas require a minimum latency period of 5-20 years. After the latency period, the risk of a sarcoma developing remains stable throughout life.

A radiation-induced sarcoma (soft tissue cancer) must meet certain requirements, set forth by Cahan and modified by Souba and associates to qualify as a true, radiation-induced malignancy:
The sarcoma must develop in a previously irradiated field
A latent period of at least 5 years must exist from exposure until diagnosis of the sarcoma
All cases must be proved histologically and be histologically different from the primary malignancy.
   The point is, screening for cancer (e.g. with mammography), as well as treating cancer, with radiation may only serve to propagate the incidence of cancer. This highlights one of the current shortfalls of conventional medicine: if we don't know what causes cancer, we risk treating it with methods that can actually cause cancer. If we know the cause of cancer, whether it is an infectious agent or chemical byproduct of an infectious agent (a mycotoxin), then we can tailor our treatments to be less radical and more specific.
Wuerthele-Caspe Livingston, V. Cancer: a new breakthrough. P. 46. 1972. Nash publishing. Los Angeles.

Many of the large, research centers, such as the Sloan-Kettering Memorial hospital in New York City, near Cornell Medical School, under Dr. Cornelius P.

Rhoads, were dedicated largely to finding a chemical or group of chemicals that would destroy the cancer cell. He would brook no competition or interference from any one who disagreed with his concepts. He was often heard to say, “When the cause and cure of cancer is found, I will find it.” He died a disappointed man. It is always amazing to me how the fallacious conclusions of a man or group of men, just because they are associated with a large institution that is heavily endowed, can sway the minds of scientists and physicians all over the world and blind them to the true facts. This was the case of Cornelius Rhoads. The Sloan-Kettering Hospital was heavily endowed with millions of dollars from private giants of industry. Rhoads wielded his authority as a heavy club. He himself was not really a scientist, but rather was a promoter and a politician determined to perpetuate the powerful cancer interest vested in him and his institution. Actually Dr Conrad Dobriner influenced much of his scientific thinking. Dr. Rhoads was committed to chemotherapy, and well he might be since he was head of chemical warfare during the Korean War. He tried to turn chemical warfare against the cancer cell within the human body. His great mistake was that he believed the cancer cell to be the causative agent of the disease and not the parasite contained within the cell. To unleash the horrors of chemical warfare and the atomic bomb in the form of a cobalt machine against the helpless victims of a microbic disease (cancer) is illogical. He was not content to limit his theories to his own institution but was determined to dictate the research policies of the entire country.

Genetics May Not Be as Important as Currently Thought

Most women with breast cancer do not have family history. American Cancer Society website (cancer.org). 1 Nov. 2001.

“Eight out of nine women who develop breast cancer do not have an affected mother, sister, or daughter,” the authors in a study reported in the Lancet (vol. 358, No. 9291:1389-1399) wrote.

This would indicate that lifestyle, rather than genes, is the more important predisposing factor when it comes to cancer.

Mycotoxins, Fungi, and Their Relation to Cancer

Wray, B B., Hayes, A. W. Aflatoxin B1 in the serum of a patient with primary pepatic carcinoma. Environmental Research. 22;400-403. 1980.

The possibility of human exposure to aflatoxins from dietary patterns in advanced societies must now be considered possible because of reports of aflatoxin B1 in persons with primary liver cancer and Reye's syndrome.

One patient (a US resident) who ate peanut butter almost daily as well as corn (grits) and colas had, among other things, liver cancer.
Gross, R., et al. Naturally occurring toxic substances in foods. Clinical pharmacology and therapeutics. 22(5 pt 2); 680-698. 1977.

All species tested to date develop liver carcinoma after exposure to aflatoxin B1. Cancer occurrences correlate with the amount of aflatoxin exposure.
Krough, P, et al. Possible mycological etiology of oral mucosal cancer: catalytic potential of infecting Candida albicans and other yeasts in production of N-nitrosobenzylmethylamine. Carcinogenesis. 8(10); 1543-1548. 1987.

Three dentists discuss how the yeast, Candida albicans, may, in fact, be responsible for causing oral cancers.
Bencini, P. L., et al. Kaposi's sarcoma in kidney transplant recipients treated with cyclosporin. British Journal of Dermatology. 118;709-714. 1988.

Once again, a mycotoxin—in the form of a drug called cyclosporin—causes cancer. Kaposi's sarcoma is a type of skin cancer that is classically seen in people with AIDS. People with AIDS are ravaged with ‘secondary’ fungal infections as their immune system progressively fails. The fungi that afflict AIDS patients are capable of producing their own mycotoxins. The relation, then, of Kaposi's sarcoma to AIDS should be no surprise; but more importantly, it should be more obvious to clinicians that mycotoxins are clearly the etiology of Kaposi's sarcoma.
Ninth Report on Carcinogens. U.S. Department of Health and Human Services. Public Health Services National Toxicology Program, revised January 2001.

Cyclosporin A is known to be a human carcinogen based on studies in humans which indicate a causal relationship between exposure to cyclosporin A and human cancer. There are numerous case reports describing cancer (mainly lymphoma or skin cancer) developing in organ transplant recipients, psoriasis patients, and rheumatoid arthritis patients treated with cyclosporin A for immunosuppression. The time between treatment initiation and tumor development ranged from as early as 1 month to 10 years.
Bernard, R. Aflatoxin showing in southern Illinois, Iowa and Ohio corn.

AgWeb.com. September 2002.

Mycotoxicoses are not third world country problems.
Mycotoxins in rice ‘low or non-detectable.’ Food standards web page. Food.gov.uk/news. September 2002.

An agency-commissioned survey of mycotoxins in retail rice has found levels in all of the 100 samples tested to be below current or proposed EC limits. The samples were tested for aflatoxins, ochratoxin A, sterigmatocystin, fumonisins, trichothecenes (deoxynivalenol, or vomitoxin), and zearalenone.

This is in agreement with CAST, 1989, which placed rice and oats among the less-contaminated staple grains (Mycotoxins: Economic and Health Risks. Report No. 116. November 1989. Council for Agricultural Science and Technology-CAST. Ames, Iowa).
Johnston, J. Aflatoxin shows up in Kansas. AgWeb.com. September 2002.

Aflatoxin has shown up in numerous Kansas corn fields recently, but says producers have options to deal with the problem. ‘This is the worst it's been since 1998,’ said Kansas State University Extension plant pathologist Doug Jardine, ‘In most cases, there are still uses for the corn.’ As many as 40-50 percent of the samples arriving at grain inspection offices at this time (1998) have detectable levels of aflatoxin, and 40 percent of those have 20 parts per billion or higher, which is the level deemed unsafe for human consumption.
Ninth report on Carcinogens. U.S. Department of Health and Human Services. Public Health Services National Toxicology Program, revised January 2001.

Alcohol is known to be a human carcinogen based on sufficient evidence of carcinogenicity from human studies that indicate a causal relationship between consumption of alcoholic beverages and cancer in humans.

Note: alcohol is a mycotoxin produced by the yeast, Saccharomyces cerevisiae.
550 sickened from Quorn fungus-based foods. CSPI Newsroom. Center for science in the public interest. Cspinet.org. May 2003.

More than 550 Britons and Americans have reported suffering vomiting, nausea, diarrhea, or anaphylactic shock after eating Quorn, the meat substitute made with vat-grown fungus. The foods are made with a mold called Fusarium venenatum—venenatum being a Latin word for ‘filled with venom.’

Fusarium species of fungi are well-known for their production of mycotoxins such as fumonisin, zearalenone, and the trichothecene mycotoxins, one of which is called, not surprisingly, vomitoxin.

Evidence for Mycotoxins Causing Genetic Damage. Also, “Tumor Markers” That are Seen in Both Cancer and Fungal Infections:

Aguilar, F., et al. Aflatoxin B1 induces the transversion of G→T in codon 249 of the p53 tumor suppressor gene in human hepatocytes. Proceedings of the National Academy of Sciences. Vol. 90, pp 8586-8590, September 1993.

Approximately half of hepatocellular carcinoma (HCC) from regions in the world with high contamination of food with the mycotoxin aflatoxin B1 (AFB1) contain a mutation in codon 249 of the p53 tumor suppressor gene.
Tsutomu, H. et al. Elevated serum CA 19-9 level and regional lymphadenopathy in a young man with allergic bronchopulmonary aspergillosis (ABPA).

A 21 year old man was suspected to have malignant neoplasm on admission to the hospital, but was diagnosed with ABPA by Rosenberg's criteria.

CA 19-9, a tumor-associated carbohydrate antigen is known to be a useful marker for GI malignancies, particularly pancreatic adenocarcinoma.
Joklik, W., ed. Zinser microbiology. Appleton & Lange. Connecticut. 1992

Aflatoxins are converted in the host (e.g. by liver microsomal enzymes) into active, unstable compounds that bind to DNA, prevent base-pairing, and induce frameshift mutations. Aflatoxin B1, the most potent liver carcinogen, also induces many other molecular changes. Other mycotoxins with proven carcinogenicity for experimental animals include ochratoxin, sporidesmin, zearalenone, and sterigmatocystin. They are produced by species of Aspergillus, Penicillium, Helminthosporium, and other ubiquitous saprophytic fungi.
Lane, K. S. Aflatoxin, tobacco, ammonia and the p53 tumor-suppressor gene: cancer's missing link? Medscape.com. Aug. 30, 1999. Medscape Portals, Inc.

Aflatoxin, the fungal carcinogen first identified in 1960, is now recognized as the prototypical laboratory carcinogen. It causes mutations in the p53 tumor-suppressor gene as well as ras mutations. Aflatoxin contamination of tobacco is not regulated by the FDA. Aflatoxin is a teratogen, mutagen, carcinogen, immunosuppressant, and potent inhibitor of protein synthesis. The p53 mutation is found in approximately half of all human cancers (56% of the time in lung cancer tissues, and between 44-50% in colorectal, esophageal, ovarian, pancreatic, and skin cancers, according to some researchers). Regarding ras mutations, in one study, lung tumors sampled from mice showed 100% k-ras mutations.
Wang, J S, Groopman, J D. DNA damage by mycotoxins. Mutat Res 1999. March 8;424(1-2):167-81.

To date, the mycotoxins with carcinogenic potency in experimental animal models include aflatoxin, sterigmatocystin, ochratoxin, fumonisins, zearalenone, and some Penicillium toxins. Aflatoxin B1 is the most potent genotoxic agent—it causes chromosomal aberrations, micronuclei, sister chromatid exchange, unscheduled DNA synthesis, and chromosomal strand breaks, and well as forms adducts in rodent and human cells. The relationship between aflatoxin exposure and development of human hepatocellular carcinoma was demonstrated by the studies on the p53 tumor suppressor gene. High frequency of p53 mutations (G→T transversion at codon 249) was found to occur in HHC collected from populations exposed to high levels of dietary aflatoxin in China and Southern Africa.
Habif, T. Clinical dermatology: a color guide to diagnosis and therapy, 3^{rd} ed. 1996. Mosby. St. Louis, Mo.

Immunosuppression leads to a great increase in the risk of squamous cell carcinoma (SCC—a type of skin cancer). Renal transplant recipients have a 253-fold increase in the risk of SCC.

Renal (kidney) transplant patients are given cyclosporin, a fungal metabolite (mycotoxin) that causes not only immunosuppression (the intended effect for someone who has just received a foreign organ so that the host body does not reject the organ), but also cancer, hypertension and high cholesterol.
Hachiya, T., et al. Elevated serum CA 19-9 level and regional lymphadenopathy in a young man with allergic bronchopulmonary aspergillosis (ABPA). Internal Medicine. Vol 37, No. 1. January 1998.

This case report describes a 21 year old man with bronchial asthma who suffered from a productive cough. A chest x-ray, taken on admission to the hospital, revealed atelectasis, pulmonary infiltrates and paratracheal and hilar lymphadenopathy. A serum CA 19-9 was elevated. He was initially suspected to have malignant neoplasms (cancer), but was later diagnosed with ABPA.

CA 19-9 is a tumor-associated carbohydrate antigen, known to be a useful marker for gastrointestinal cancers, particularly those of the pancreas. Elevated levels may also be seen in cancers of the lung, as well as non-cancerous conditions such as idiopathic pulmonary fibrosis, bronchiectasis, diffuse panbronchiolitis, and cystic fibrosis.
Dorrenhaus, A., et al. Induction of unscheduled DNA synthesis in primary human urothelial cells by the mycotoxin ochratoxin A. Toxicol Sci. February 2000. 53(2):271-7.

Ochratoxin A is a widespread contaminant in human staple food. Exposure of humans to this mycotoxin is a matter of concern because ochratoxin A is a known rodent carcinogen.
Costantini, et. al. Etiology and prevention of prostate cancer: Hope at Last. Fungalbionics series. 1998/1999. Johann Friedrich Oberlin Verlag. Freiburg, Germany.

The PSA is a 33-kDalton serine protease inhibitor made by the Ascomycete fungi, Aspergillus flavus, Aspergillus fumigatus, Aspergillus oryzae, Ophiostoma piceae, and Scedosporium apiospermum. An elevated PSA is seen not only in men with prostatic disease, but also in women with breast, ovarian, pancreatic, and colon cancer; and even in women during pregnancy.

There has been much, recent confusion on exactly what is the role of the prostate specific antigen (PSA) in screening for and following the course of prostate cancer. Up to 50% of the time, a “positive” (high) PSA level, upon further, biopsy evaluation, does not correlate with prostate cancer. If PSA screens for fungus, then PSA levels should decrease with antifungal therapy, hence:
Mann, D. Antifungal agent lowers PSA levels, study finds. May 1, 1997. p6. Medical Tribune.

The antifungal drug, Nizoral® (ketoconazole) lowered PSA levels in men with prostate cancer. It was suspected that ketoconazole's ability to increase estrogen levels, via inhibiting the breakdown of estrogen in the liver in men, was the mechanism for this lowering effect. However, if indeed a high PSA signals a fungal infection gone awry, then the antifungal effect of ketoconazole should be the obvious reason for the lowering of the PSA.

Mycotoxicoses are not Rare, as is Typically Thought to be the Case

Arena, Jay; Drew, R. Poisoning: Toxicology, Symptoms, Treatments. 5^{th} ed. Charles C Thomas. Springfield, Ill. 1986.

Mycotoxins may turn out to be responsible for more than one human ailment about which current textbooks say “pathogenesis unknown.”

Americans consume an estimated 0.15 to 0.50 mg of Aflatoxins daily. Grains, peanuts tree nuts and cottonseed meal are among the more common foods on which these fungi grow.
Wren, W. Aflatoxins high in state's central region. Fort Worth Star Telegram. Aug. 6, 1996.

Corn and grain sorghums rejected for human use because of high aflatoxin levels are going to feed lots for livestock.
American Institute for cancer research newsletter. Issue 63. Spring 1999.

Cancer risk may be increased by drinking any amount of alcohol, and it doesn't matter if it's beer, wine or whiskey.
Aflatoxins. U.S. FDA Center for Food Safety and Applied Nutrition. Foodborne Pathogenic Microorganisms and Natural Toxins Handbool. Vm.cfsan.fda.gov. April 2001.

Aflatoxins produce acute necrosis, cirrhosis and carcinoma of the liver in a number of animal species. No animal species is resistant to the acute toxic effects of aflatoxins. Aflatoxicosis may be suspected when a disease outbreak exhibits the following characteristics:
The cause is not readily identifiable
The condition is not transmissible
Syndromes may be associated with certain batches of food
Treatment with antibiotics or other drugs has little effect
The outbreak may be seasonal, i.e. weather conditions may affect mold growth.
Regrettably, clinicians rarely ask these questions.

In the United States, aflatoxins have been identified in corn and corn products, peanuts and peanut products, cottonseed, milk, and tree nuts such as pistachios, brazil nuts, pecans, and walnuts. Other grains and nuts are susceptible but less prone to contamination. The above characteristics of a mycotoxicosis rarely, if ever, are addressed in a typical physician's office during a routine patient visit. For example, if an antibiotic does not work for a particular illness, rather than thinking of a fungal or mycotoxin etiology of the disease, a ‘stronger’ antibiotic is typically prescribed.

Diet and Cancer—Studies that Part From Mainstream Thought

Stenson, J. High-fat diet may not increase the risk of breast cancer; carbohydrates cited. The Medical Tribune. Jun. 20, 1996. Vol 37, No. 12.

If, per the January 2002 JAMA article (Etzel, R. Mycotoxins. Journal of the American Medical Association. 287(4). Jan. 23/30, 2002.), mycotoxins are common contaminants of grains, and if grains often are tainted with carcinogenic mycotoxins, this might explain why grains were cited as an increased risk factor for breast cancer.

A study reported in the Lancet (1996;347:1351-1356) stated that a high fat (unsaturated fats) diet was associated with a lower risk (19% lower) of breast cancer than a low-fat diet, while starch and carbohydrates were found to raise (by 30-39%!) breast cancer risk. The researchers called the link between starch and breast cancer ‘difficult to explain.’ The study was said to be ‘well-done.’
Toth, B, et. al. Cancer induction in mice by feeding of the uncooked cultivated mushroom of commerce, Agaricus bisporus. Cancer Research. 46;4007-4011. August 1986.

Apparently, even the common, table mushroom causes cancer. It is, however a fungus and not a vegetable, as A. V. Costantini, MD once said.

Evidence for the (Ascomycete) Fungal Spore's Role in Cancer

White, M. W. Plant bacterial spores, active systemically as a separate entity, play a significant role in human illnesses such as cancer, granulomas, AIDS and milky white abdominal ascites that currently defies recognition. Medical Hypotheses. 44;493-503. 1995.

When malignant tissue is removed and examined as with an H&E stain, or a Pap smear, or even under the electron microscope, a diagnosis is readily made for pathology but a diagnosis for origin is clearly missed because the stains, the fixatives, plus the atmospheric oxygen contribute completely to eliminate the spore's presence or disarranges their appearance for a proper recognition.

The major source of entry of spores systemically in human beings is via the alimentary tract s a result of the ingestion of moldy goods, moldy cheeses, etc.

Spore entrance may occur as well by inhalation, damaged skin, or mucosal linings. Transfusions may also be a factor if they have the spore already present.

In the malignancies, the spore of the Ascomycete is required, because the latter, with its genetic viable oxidant factor, can survive within a sac or cell. The benign granuloma will develop from the presence of spores arising from the non-Ascoymcete family. The spores stemming from the facultative microorganisms, which has genetically both animal and plant genes, will or can lead to AIDS.
White, M. W. A Specific Oxidant is the Prime Factor in Cancer Cells' Origin and Growth. Medical Hypotheses. 42;313-317. 1994.

Anaerobia (metabolism without oxygen) in malignant growth activity is not a new concept for cancer activity. Otto Warburg, a British scientist, and Yoshicki Okmoto compiled a number of articles into a textbook format and labeled the book Metabolism of Tumors. This book was published in 1930 by Arnold Constable, located in London, England.
White, M. W. Cancer is a hybrid produced by a relationship between a plant bacterium and a mammalian cell: an original concept. Medical Hypotheses. 1996. 47;35-38.

Ultimately, with an ensuing circulating but compatible flow of blood by the host, there develops an annealing process whereby the genes of each species unite to form a plant-animal intracellular hydridization.

Because of the anaerobiosis, there is an incomplete glycoslysis (breakdown of sugar) at the inflammatory site involved. This contributes to an excessive accumulation of the intermediates as the various phosphates, the glyceraldehydes, and high levels of lactic acid. There is also an incomplete lipolysis (breakdown of fats) with the associated acidosis. Proteolysis (Krebs citric acid cycle) is similarly inhibited with the accumulation of the toxic levels of ketoglutaric, oxaloacetic, succinic, and lactic acids as well. In consequence, there is an accumulation of the chronic defense cells, the squamous and/or epithelial cells, etc. depending on the site involved. Because of the unresolved pathophysiology, to this anaerobic inflammatory response there is thus the ultimate stymied repair mechanism and the continued progress of the growth of the diseased tissue (i.e. tumor growth).

Fungi can metabolize anaerobically—similar to cancer cells—and produce lactic acid as a byproduct. This adds fuel to the argument as to whether a cancer cell is actually a cell that has become infected with a fungal organism, because no normal, human cell can metabolize anaerobically for long periods of time.
White, M. W. Pathway to carcinogenesis: the role of bacterial spores. Medical Hypotheses, 35:279-287. 1991.

There are plant bacterial spores present within the malignant cell. It is important to emphasize that it is not any plant bacterial spore, but only the ones that arise not only as the primitive asexually reproductive conidial or unicellular form from the budding or branching adult microorganism, but also one genetically capable of surviving, despite an absent or deficient outside cell wall, and the loss of many of their original enzymes and metabolites, snugly and safely within a sac or cell along with its reducing or ‘deoxygenating’ capability. The Ascomycete fungi belong to this class of plant-like bacterial activity and can produce such spores when in duress.

These spores can be visualized within the malignant cell but only if studied cytobiologically as a wet smear.

In the medical literature bacteria or viruses have been seen or recovered in in vitro studies. However, their mere presence is not a criterion for an etiological connection. There must be a pathway of connecting activity to relate the presence or recovery of a microorganism from cancerous tissue as an etiological factor or cofactor. The Ascomycete fungi demonstrate such a pathway.
White, M. W. Increasing evidence that the “transformed” yeast or mold microorganism is the etiological factor behind the pathogenesis of cancer tissue. International Surgery. Section 1, Vol 48, No. 5. November 1967.

New (at the time) experimental evidence increases our conviction that there occurs a transformation of yeast or mold microorganisms, under certain circumstances, into a parasitic, anaerobic, unicellular, oval conidial form, and that these transformed cells then assume the characteristics which strongly stimulate the eventual formation of the cancerous cell. Interest in this area of study was reestablished (after initial suspicions of the role of fungi in cancer in the 1930's) when it was observed how turkeys that ate mold-infested grain developed malignant tumors. However, in both old and new cases, since fungal organisms could not be isolated from the tumors, Koch's postulates could not be satisfied. (Koch's postulates outline the necessary qualities and abilities of a disease to be classified as an infectious disease, and to establish a germ as a cause of an infectious disease. Cell wall deficient fungal spores, related to the process of carcinogenesis, cannot completely fulfill Koch's postulates—since these altered fungal spores defy the postulates). Yet, in this study, the researchers were able to achieve retransformation of yeast or mold (from fungal spores) from cancerous material in a high percentage of cases. The fungal spores that were studied in this and in future papers—the ones isolated in cancerous tissue—were actually altered fungal spores: they were deficient of the normal, protective cell wall. Without the cell wall, they could not survive alone, outside of a host (human) cell; therefore they were “obligate parasites.” Similarly, they could not stand up to the normal, pathological and microbiological techniques used to study cancerous tissue, like, for example, the submersion of the cancer tissue specimen into formalin. If special isolation precautions and culturing techniques were not used—those techniques outlined by this author in later papers—the cell wall-deficient fungal spores would never be seen or cultured, and therefore would never be suspect as an infectious cause of cancer.
White, M. W. The reducing activity of cancerous tissue and its apparent significance. The journal of abdominal surgery. Vol 15, No. 10. October 1973.

One cannot escape the irrefutable evidence that cancer acts like a plant growth in an animal environment; in that the end acceptor is H+ ion oriented in preference to O2. The assimilation of the conidia (from the fungus) by the defense cell (macrophages, etc) with retained viability constituted a malignant cell.

More simply said, the malignant cell is defined here as an immune cell that has been invaded by an altered, parasitic, fungal spore.
White, M. W. Metabolism of the malignant cell, in vivo (in the body) is anaerobic and significantly plays a factor in the pathway to carcinogenesis. Medical Hypotheses. 39;323-333. 1992.

The malignant cell, in vivo, metabolizes and respires anaerobically (can live without oxygen). For a good many years scientists have been aware of anaerobia being present in malignant growths.
White, M. W. Cancer: the role of oxygen in fungal induced carcinogenesis. Medical Hypotheses. October. 2000.

“I have come to a definite conclusion that cancer is a chronic, infectious, abnormal, anaerobic, respiratory and metabolic “germ” disease; the germ being an invasive, obligate (parasitic) asexual anaerobic (spore), a member of the Ascomycete group of fungi.

Anaerobiosis is defined as cellular life without the ultimate oxygen molecule. The mammalian cancer cell demonstrates anaerobiosis, but as evidence indicates, the cancerous animal cell not only survives but grows and spreads as well.
Cancer following germ injection. Science News. Science Service, Washington, D.C. vol 77, No. 1997. Apr. 7, 1933.

Development of cancer following the injection of a germ or micro-organism is announced by the U.S. Public Health Service's National Institute of Health. The discovery was made by Drs. T. J. Glover and J. L. Engle, who have been working at the institute, although they are not attached to the regular government staff nor to the U.S. Public Health Service. They have succeeded in producing typical, unmistakable cancer in a guinea-pig. This cancer followed the injection of a culture of a micro-organism or germ isolated from the tissues of a proved case of cancer of the human breast. This traverses the prevalent opinion that cancer is not a germ disease. It is only after years of work that the announcement has been made. The germ itself is what scientists call a spore-bearer. It was isolated on a special media from the tissues of the human cancer.

“Proper” Identification of Fungal Spores in Cancerous Tissue

White, M. W. A Specific Oxidant is the Prime Factor in Cancer Cells' Origin and Growth. Medical Hypotheses. 42;313-317. 1994.

Recovery of adult fungi: If the malignant tissue is freshly collected aseptically and immediately placed in a Ringer's lactate solution containing EDTA as an anticoagulant and then placed in a Sabouraud's Dextrose agar tube with a tightly closed cap, it may be possible, if the spores within the malignant cell have sufficient remnant metabolites and enzymes to recover them not as spores but as newly formed adult microorganisms.

These spores in their transformation from their original adult form sustain not only a loss of most of their original enzymes, but also a loss of their budding or branching reproductive capability, and a consequential deficient or absent outside cell wall. They retain however their ability to reproduce asexually and metabolize anaerobically. They eventually form the cytochrome and prothrombin elements derived from the compatible circulating blood flow of the host.

If the cancer cell is prepared as in an H&E stain these unicells (cell-wall deficient spores) are dissolved and consequently will not be present or seen. Even with electron microscopy their appearance can be misinterpreted.
White, M. W. Metabolism of the malignant cell, the role of bacterial spores, and a pictorial presentation to substantiate the latter's presence as an etiological factor in carcinogenesis. Medical Hypotheses 39;95-109. 1992.

Visualization of the spores: They must be viewed as a wet smear—a piece of cancer tissue is freshly removed and placed in 10 cc of Ringer's lactate solution containing 10.5 mg of EDTA. The tissue is gently minced and a drop of it is placed on a glass slide, covered with a cover slip and studied under the microscope cytobiologically. These oval and spheroidal unicells are present within the malignant cell varying in size from minute to 1-2 microns in diameter, and varying in numbers.

It is useless to use the H&E, Pap, and Gram stains for spore identification.

The Prevalence/Non-Recognition Problem of Fungal Infections and Mycotoxicoses

Joklik, W., ed. Zinser microbiology. Appleton & Lange. Connecticut. 1992.

Since the mycoses are not reportable diseases, their prevalence is unknown.
Van Egmond, H P. Advances in experimental medicine and biology. Medline citation ID #21919057. May 2003.

It appears that 77 countries now have specific regulations for mycotoxins. Thirteen countries are known to have no specific regulations, whereas no data are available for about 50 countries, many of them in Africa.

Of those that have regulations, such as the United States, some only have regulations for a single mycotoxin—aflatoxin. This, despite the common presence of many other harmful mycotoxins that are found in our staple grain foods.
Fraser, D., et al. Aspergillosis and other systemic mycoses: the growing problem. Journal of the American Medical Association. Oct. 12, 1979. Vol. 242, No. 15.

Marked increases from 1970 to 1976 were found in the incidence of aspergillosis (158%), actinomycosis (92%), cryptococcosis (78%), and coccidioidomycosis (74%).

Question: did this ‘growing problem’ just go away? If we've prescribed more antibiotics since then, and if AIDS is now present, as it was not at that time, one could only guess that the problem has been skyrocketing since 1976. Yet, who can be certain, since systemic fungal infections remain non-reportable diseases (i.e., there is no requirement to report these fungal infections to the CDC). Indeed, the incidence has increased: by 1996, there were an estimated 10,190 aspergillosis-related discharges from U.S. hospitals, an eightfold increase from the 1976 figure
(Warnock, D. et al. Epidemiology and prevention of invasive aspergillosis. Current infectious disease reports. 2001,3:507-516).
Chaturvedi, V. Coccidioidomycosis in New York State. Emerging infectious Diseases 6(1), 2000. Centers for Disease Control.

This fungus, endemic to the southwestern United States, has found its way across the country, if not the world. An estimated 100,000 infections occur annually in the U.S., and 0.5% progress to systemic infections. Increased travel has caused the ‘spread’ of these area-confined fungal infections; thus it is no longer feasible to rule out a fungal etiology in the workup of a disease or illness based on the current location of the patient and hospital alone. The travel history of the patient must be considered in the work-up of a particular disease.
Also: Lin, J.; Hamill, R. Coccidioidomycosis Pulmonary Infection. Current Infectious Disease Reports 2001,3:274-278.

As a result of the increased mobility of the population (particularly the elderly), tourism, troop turnover from military bases, and the exchange of raw materials for manufacturing and construction, cases [of coccidioidomycosis] have been reported with increased frequency from non-disease-endemic areas.
Baldwin, Richard S. The Fungus Fighters: Two Women Scientists and Their Discovery Cornell University Press. Ithaca and London. 1981.

Without these vital statistics [gained from requiring fungi to be reportable diseases to the Centers for Disease Control], support for medical mycological teaching, training and diagnostic centers, as well as basic and applied research, is difficult to justify and funding difficult to obtain from administrators. Medical mycologist must compete for support from a limited pool of funds against investigators of all other diseases. But the others [bacterial and viral illnesses], being notifiable [to the CDC], are backed up by data on morbidity and mortality that sway the minds of men and loosen purse strings.

Baldwin stressed that “physicians are the key figures in any attempt to get better data on the possible public health implications of the fungus diseases, yet there is no federal law requiring the reporting of these diseases to the CDC. Even more, the individual states have their own laws as to which diseases will be reported within the state (P. 199).”

The information is lacking not just in the United States. Speaking before the Oholo Biological Conference in Maalot, Israel in 1976 Ajello of CDC said that 1976, no country in the world has made mycotic (fungal) diseases notifiable to a public health agency.

The NIAID made 2 grants to fund centers for medical mycology—UCLA and Washington University at St Louis under the recommendations of its 1977 Workshop on Medical Mycology Research and Training (p.194). The American Society of Microbiology (ASM) News touted, “The creation of these units reflects recognition, by NIAID, that fungal infections have become an increasingly important cause of disability and death in this country (didn't they say that in the 1940's?). The ASM went on: ‘Ironically, the emergence of this problem reflects the darker side of new treatments for malignant or immunological disorders; such treatments often appear to weaken the defense mechanisms that ordinarily prevent such infections’. This means that our newer medical treatments, including antibiotics, were actually contributing to the problem. If this was such a problem from the 1940's to 1977, and therefore must certainly be an even bigger problem today, then why do the medical mycology programs at UCLA and Washington University not exist anymore?

It was noted that “as to the physicians, in many medical schools the curriculum did not include lectures in medical mycology in others the microbiology course might have three lectures which cover the entire field of mycology. In 1970, most medical technologists and public health microbiologists received no training in medical mycology. Those who happened to be specializing in mycology were concentrating largely on the nonpathogenic varieties of fungi—those concerning plants and insects or perhaps the edible fungi.”

Libero Ajello, director of the Mycology Division, Laboratory Bureau, of the CDC in Atlanta in the early 1970's stated, “Any attempt to quantitate the impact of the mycoses on public health is doomed to failure. Since they are not classified among the notifiable diseases, hard data on their incidence and prevalence, as well as information on the morbidity and mortality they cause, are either fragmentary or simply not available (p.30).” In 1969, the Centers for Disease Control (CDC) began to gather, organize, and publish data voluntarily supplied by physicians and investigators around the country who maintained their own records on fungal diseases. Four years later this effort came to a halt when funds for CDC were cut, another casualty being the closing of its Kansas City field station which had been outstanding as a research and training center for medical mycology, and as a sponsor of similar programs in other institutions (p.31).
Conant, et al. Manual of Clinical Mycology. 2^{nd} ed. WB Saunders Co. Philadelphia, 1954.

Fungus infections are of such common occurrence that we have found it necessary to consider mycotic disease in the differential diagnosis of practically every obscure infection.

And . . .

Fungus infections are relatively, if not actually, more frequent in occurrence since the introduction of penicillin and other potent antibiotics for the control of the acute bacterial diseases.
John Rex, MD. Managing fungal infections in the new millennium. Medscape.com. April 2000.

“A wide variety of fungi now isolated from neutropenic patients were not previously recognized as human pathogens. Many of these are soil or plant fungi—organisms that clinicians have not been trained to recognize.”

And . . .

“There are no, rapid, accurate diagnostic tests that canconfirm with certainty the presence of invasive fungal disease.”
C. C. Kibbler, et al. Principles and Practice of Clinical Mycology. John Wiley and Sons. Chichester. 1996.

“The escalating incidence of these infections is linked in part to the widespread use of broad-spectrum antibiotics and the advent of increasing numbers of patients with cancer and other underlying diseases receiving intensive immunosuppression regimens.
Burrow, W. Textbook of Microbiology. W.B. Saunders Co. Philadelphia 1959.

In spite of its earlier beginnings, medical mycology was soon overshadowed by bacteriology and has never received as much attention, though some of the fungous disease are among the more common infections of man.

Mike Rinaldi, PhD, director of the fungus testing laboratory in the department of pathology and professor of pathology, medicine, and clinical lab science at the UTHSC at San Antonio, in an article by Hellinghausen, M. Fungal infections pose danger. Nurseweek. 22 Apr. 1996.

“We've reached the point where fungal disease can't be considered a minor problem. Invasive candidiasis has become the fourth leading cause of hospital-acquired infection.”
Peraica, M., toxicologist, Unit of Toxicology, Institute for Medical Research and Occupations Health, Ksaverska cesta 2, POB 291, HR-1001 Sagreb, Croatia, et al. Toxic effects of mycotoxins in humans.

“Mycotoxicoses often remain unrecognized by medical professionals, except when large number of people are involved.”

Also . . .

“The toxic effects of mycotoxins (e.g. ochratoxins, fumonisins, zearalenone, etc.) are mostly known from veterinary practice.”

Also . . .

Mycotoxicoses are usually insufficiently treated in medical textbooks and are not covered in curricula of many medical schools.”

Infectious Disease Characteristics of Cancer/Characteristics that Cause Clinicians to Confuse Fungal Infections for Cancer:

Islam, A. The origin and spread of human leukemia. Medical Hypotheses 39;110-118. 1992.

The human leukemias are presumed to be clonal diseases, arising from an alteration in a single, hematopoietic stem cell, which then proliferates and replaces the marrow of normal hematopoietic stem cell systems. Results of our current morphologic studies on well-fixed, ideally-stained thin sections of plastic-embedded bone marrow biopsies from a large number of acute and chronic leukemia patients suggest that human leukemias may not be clonal diseases. Instead, a large population of other resident cells—“endosteal cells”—appears to become involved in the process and it is possible that all members of this group enter the activity simultaneously. This change (transformation) in the endosteal cell population might be due to an abnormality (qualitatively or quantitatively) of diffusible, humoral factors (yet to be identified) that are responsible for the growth and proliferation of these hematopoietic precursor cells. In this context, the human leukemias may be considered not as malignant, but rather the result of an aberration of factor(s) that control hematopoiesis (the formation and development of blood cells in the bone marrow).

This study is suggesting that leukemia is not a malignancy, but rather a condition that arises as a result of an insult to the bone marrow. Cancer chemotherapy drugs—often derived from fungi—as well as the trichothecene mycotoxins are very well-known for their ability to cause bone marrow abnormalities.
Comprehensive Cytopathology, 2^{nd} ed. W.B. Saunders Co. Philadelphia, Pa. 1997.

Calcium oxalate is a metabolic product of Aspergillus spp., especially Aspergillus niger. The presence of such crystals in a background of inflammatory cells should be a clue to infection with A. niger.

Microcalcifications, a clue to malignancy seen in a mammogram, could be due to an Aspergillus niger infection and not cancer. If a fungal infection is never thought of when calcifications are seen on mammography, a search for such infection will never be carried out, and the erroneous treatment for ‘cancer’ will ensue.
Nosanchuk, J D, et al. Histoplasma capsulatum synthesizes melanin-like pigments in vitro and during mammalian infection. Infect. Immun. September 2002; 70(9):5124-31.

Melanin is made by several important pathogenic fungi and has been implicated in the pathogenesis of a number of different fungal infections. Melanin is an important virulence factor in other pathogenic fungi, and may have a similar role to play in the pathogenesis of histoplasmosis.

Question: can a systemic infection with Histoplasma capsulatum, or other melanin-producing fungi, look exactly like metastatic, malignant melanoma? By this article, it would appear to be possible.
Treatment of fungal infections led to leukemia remissions. Medical Tribune, Sep. 29, 1999. intellihealth.com. October. 1999.

In a study presented September 28, 199 at the Interscience Conference on Antimicrobial Agents and Chemotherapy in San Francisco, Dr. Meinolf Karthaus, associate professor of Hannove Medical School in Germany, reported that the administration of three antifungal medications to three patients with leukemia not only cured their (secondary) fungal infections, but successfully treated their cancer as well. The patients received amphotericin B, as well as high doses of fluconazole and liposomal amphotericin B. The main message, said Karthaus, is that if patients have severe fungal infections, then treatment has to be started for these patients. Physicians shouldn't give up on them.”
Hachiya, T., et al. Elevated serum CA 19-9 level and regional lymphadenopathy in a young man with allergic bronchopulmonary aspergillosis (ABPA). Internal Medicine. Vol 37, No. 1. January 1998.

This case report describes a 21 year old man with bronchial asthma who suffered from a productive cough. A chest x-ray, taken on admission to the hospital, revealed atelectasis, pulmonary infiltrates and paratracheal and hilar lymphadenopathy. A serum CA 19-9 was elevated. He was initially suspected to have malignant neoplasms (cancer), but was later diagnosed with ABPA.
Hennenfent, B. Letter to the editor: Prostatitis and benign prostatic hypertrophy: emerging infectious diseases? Emerging Infectious Diseases. Cdc.gov. November 2001.

In these days of prostate specific antigen testing, more than 50% of men who undergo biopsies for prostate cancer have a prostatitis lesion whether they have cancer or not. Prostatitis as a histologic lesion has been found in 98% of patients with benign prostatic hypertrophy (BPH).

If our body uses inflammation to fight off infectious germs, then could an infectious germ be causing not only BPH, but also prostate cancer? Candida albicans and Blastomyces species of fungi are known to infect the prostate. In addition, Costantini, et al has already outlined the studies showing that the PSA is a molecule produced by the Ascomycete group of fungi. A “positive” PSA test in a male would, by this measure, indicate not cancer, but a fungal infection!
Davidson, S., et al. The Principles and Practice of Medicine: a textbook for students and doctors. The Williams and Wilkins Company. Baltimore, 1967.

A large mass of mycelium in a pulmonary cavity, often described as an ‘aspergilloma’ may simulate a tumor on radiological examination.
Medscape.com. May 2003. Encephalitis, E. coli in New York, Resistant Staph in Midwest, GI fungal infection in Arizona.

“Because basidiobolomycosis is an unusual fungal infection, often found in association with amphibians or reptiles and their excrement, it can easily be misdiagnosed.”

This report outlines two cases of gastrointestinal basidiobolomycosis, one of which was thought, by appearance on CT scan, to be gastric (stomach) cancer in a 37 year old woman, and the other thought to be colon cancer in a 59 year old man. Both patients underwent surgery directed at removing these “tumors.” It was only after further pathological testing of the removed portion of the organs that a basidiobolomycosis fungal infection was diagnosed. Both patients recovered well after receiving appropriate antifungal therapy.
Kauffman, C. Nonresolving pneumonia: is endemic mycosis to blame? The Journal of Respiratory Diseases. Vol 16, No 11. November. 1995.

Acute pulmonary Blastomycosis mimics many other types of pneumonia, while chronic Blastomycosis may cause cavitary or mass-like lesions, which may be misdiagnosed as lung carcinoma.

If this fungal infection can be misdiagnosed as lung cancer, and if our techniques for isolating fungi are inadequate, and if physicians are poorly trained to recognize fungal infections, what keeps a person with a Blastomycosis mass in the lung from going on to be treated with chemotherapy?
Kibbler, C., ed. Principles and Practice of Clinical Mycology. John Wiley and Sons, Chichester. 1996.

Regarding Blastomycosis and bone infections: Plain radiographic findings may be similar to those in tuberculosis, metastatic neoplasm, rheumatoid arthritis, sarcoidosis, eosinohpilic granuloma and pigmented villonodular synovitis. (p51).
Differention of blastomycotic bone disease from tuberculosis, malignant disease or other fungal disease is difficult. (p53)
The macroscopic appearance of tissues in cases of chronic Blastomycosis are typically hyperplastic and may be confused with carcinomatosis (metastatic, or diffuse, cancer), in contrast to the suppurative lesions seen in disseminated infection.

Gastrointestinal infections caused by dimorphic fungi (in this case Histoplasma capsulatum:

The common lesions [caused by these fungi, and seen in the intestines] were masses or ulcers mimicking inflammatory bowel disease or carcinoma.

Gastrointestinal infections caused by opportunistic molds (in this case, zygomycosis):

In a recent review of gut manifestations, chronic peptic ulcers were invaded by zygomycetes in 10 patients (Thomson et al., 1991). In nine of these 10, laparotomy (open surgery on the abdominal cavity) was required because of ulcer complications and in the tenth the patient's pyloric stenosis resolved spontaneously. In the nine patients undergoing surgery, the ulcer was usually thought to be malignant because of its hardness and the penetration of adjacent structures. Similarly, barium meals were suggestive of malignancy in seven but the diagnosis was revealed following endoscopy and biopsy.

Blastomycosis of the Respiratory Tract:

Blastomyces dermatitidis causes self-limiting respiratory infection which presents as localized pulmonary lesions in immunocompetent [normal] patients. The infiltrates of pulmonary Blastomycosis appear as a bronchopneumonia or segmental consolidation. These lesions in non-immunocompromised patients may persist for several months and lead to evaluation for chronic pneumonia or pulmonary neoplasm.

Concomitant cutaneous lesions may be ulcerative or verrucous [wart-like] and resemble a variety of chronic infections or skin cancer.
The pseudoepitheliomatous hyperplasia and desmoplastic reaction of pulmonary blastomycosis may simulate bronchogenic squamous cell carcinoma. Unless special stains for fungi are utilized on such tissue, conventional H&E stains may not detect the presence of [fungal] organisms. The presence of concomitant bony lesions may lead to an erroneous diagnosis of squamous cell carcinoma of the lung with bony metastasis unless culture and special stains are performed. (p254)

### EXAMPLES

### A. Examples of Cancer Being Treated and Recurrences Being Prevented by Antifungal Measures, Using Both a Low-Carbohydrate Diet and Natural/Prescriptive Antifungals

1. A 62 yr. old female who had a history of hormone replacement therapy (HRT) use for 14 yrs, as well as a history of recurrent tonsillitis, treated with antibiotics, was diagnosed with “aggressive breast cancer” upon biopsy of a lump discovered by her and her doctor in April of 1999. The risk of fungal and yeast overgrowth following the use of estrogen, progesterone, and antibiotics is well documented.

Her cancer was negative for estrogen receptors. Her recommended course of treatment by one oncologist was chemotherapy, surgery and radiation. Another oncologist recommended at least surgery and radiation. She had a lumpectomy done in April of 1999. Greater extension of the mass was noted at the time of surgery, so she underwent a complete mastectomy in May of 1999. She had 10 lymph nodes removed, which were all free of cancer.

She opted not to follow up with chemotherapy and radiation, against the advice of her oncologists, and made some lifestyle changes instead. These changes consisted, in part, of a low-grain diet (high in vegetables, fish, nuts, some fruits, and meats) that she began to follow. She had a PET scan done in early 2003 and showed no recurrence of cancer.

2. A 68 year old male with a history of diabetes (diagnosed 5 yrs prior to the cancer) and a 30 year history of smoking (pipe and cigars).

He began eating a 5 pound bag of in-the-shell peanuts every week starting in 1997. By February of 2000, he notice large, marble sized lumps in his neck. He went to the VA Hospital in St. Petersburg, Fla., where, upon biopsy and pathological evaluation, it was suspected that he had cancer of the lymphoma type. The specimen was sent to a lab at the National Institutes of Health, where “Large B-cell Lymphoma” was verified. No other sites of cancer were detected upon radiologic evaluation—his chest and abdomen were free of any discernable masses.

The treatment recommended by his oncologists was surgery, chemotherapy, and radiation. He and his wife decided to do none of these. Having heard of the aflatoxin contamination potential in peanuts, his wife took him off all peanuts and peanut products and began to follow a low-grain diet. In addition, she place him on several, natural antifungal substances, such as Silymarin (milk thistle), Essiac tea (an herbal tea blend), an “anti-plague formula” (an aged, extract of a blend of fresh onions, garlic, horseradish, ginger, vodka, hot peppers), and vitamin C. Garlic, vitamin C, and many herbs exhibit well documented antifungal effects.

Upon instituting these lifestyle changes, the masses in his neck began to “soften” within a week, and they completely resolved within a month. They have yet to recur in over 3 years since.

3. A 49 year old female with a strong family history of various types of cancer. Her history includes a brief use of oral contraceptives in her 20's, as well as some complications that followed her pregnancy in 1991 which were as follows: she had slow progression of her labor (over 3 days), prolonged rupture of her membranes, which always spurs the use of prophylactic antibiotics, and an eventual, emergency C-section.

One month after her pregnancy, her ankles began to swell. This spontaneously resolved, but returned in September of 1991. Initially this was treated with Amoxicillin, but upon further analysis, it was found that she had a type of kidney disease known as glomerulonephritis. In addition, she had congestive heart failure and an enlarged liver. Subsequently, she was on prednisone for 3 years (for treatment of the kidney disease) as well as cytoxan, diuretics, coumadin, and various, other drugs.

The potential for opportunistic fungal infections to take hold during times of treatment with antibiotics and corticosteroids is well documented.

She was able to wean off of the steroids after 3 years.

By 2001, she was diagnosed with colitis and irritable bowel syndrome. Intestinal dysbiosis due to antibiotic use is also well documented.

In October of 2001, she passed out due to severe anemia. At her doctor's, her hemoglobin was found to be less than 4 g/dl. She was referred to the hospital and had a blood transfusion. In the search for the etiology of her anemia, a very large, intestinal mass was discovered both on exam and radiologic workup. Undergoing surgery, she had a large, grapefruit-sized mass, along with a portion of her small intestines, removed. Pathological evaluation of the mass revealed “moderately to poorly differentiated adenocarcinoma versus malignant carcinoid tumor, with extension into the intestinal adipose tissue.” Three out of nine lymph nodes removed from the abdominal cavity were also positive for cancer involvement.

Adjuvant therapy recommended by her surgeon and oncologist was combination, low dose chemotherapy.

Instead of doing chemotherapy, she decided—against her doctors' advice—to follow a low-carbohydrate diet and take various, natural antifungal substances—caprylic acid and garlic. In addition, she took shark cartilage, which has anti-angiogenesis properties.

By December of 2001, her blood and tumor markers were normal, and remain so almost 3 years later.

4. A 60 year old man was diagnosed as having prostrate cancer. UTSW study for BPH ×5-6 yrs. Biopsy prior and post program. Normal PSA! At post-study diagnosis, cancer was found: October 2001. He moved to OKC and started watching Doug's show.

Past history includes heavy use of antibiotics, recurrently for allergies, sinusitis.

Went to MD Anderson—found cancer in different place of the prostate, but Cancer was confirmed nonetheless. Gleason stage 7.

Started on Phase I for 2 months (had severe die-off ×4 days—including fever), nystatin thereafter ×2 months. Cont'd diet for 4 months and stays on a variant of the diet. On UGN, Caprylic acid, GSE now ×3 months. Saw John Hogan, D.O. (OKC). Past 2 yrs: PSA now is 1.1. Was closer to 4.0 at the time of cancer diagnosis.

F/U Ultrasound: cannot find any tumors. Whole body scans have been negative.

### B. Examples in the Literature

Treatment of fungal infections led to leukemia remissions. Medical Tribune, Sep. 29, 1999. intellihealth.com. October. 1999.

In a study presented Sep. 28, 1999 at the Interscience Conference on Antimicrobial Agents and Chemotherapy in San Francisco, Dr. Meinolf Karthaus, associate professor of Hannove Medical School in Germany, reported that the administration of three antifungal medications to three patients with leukemia not only cured their (secondary) fungal infections, but successfully treated their cancer as well. [The patients received amphotericin B, as well as high doses of fluconazole and liposomal amphotericin B.
Mann, D. Antifungal agent lowers PSA levels, study finds. May 1, 1997. p6. Medical Tribune.

The antifungal drug, Nizoral® (ketoconazole) lowered PSA levels in men with prostate cancer.

Diet and Fungal Infections/Mycotoxins

Conant, et al. Manual of Clinical Mycology. 2^{nd} ed. WB Saunders Co. Philadelphia, 1954.

A low carbohydrate diet is necessary and the weight should be reduced if obesity is present. Occurrence of candidiasis during antibiotic therapy should not be disregarded. All antibiotic therapy should be stopped regardless of the nature of the primary infection.

Cancer That can be Treated/Prevented With Antifungals (Prescriptive Drugs as Well as Antifungal Supplements)

Charnon, Jody. Selenium supplements reportedly lower the risk of some cancers in humans. Medical Tribune. Vol 38, Number 2. Jan. 23, 1997.

May lower the risk of prostate, lung and colorectal cancer. (50% lower risk of dying from the cancer and 37% lower risk of developing cancer).

Why does selenium lower the risk of some cancers?

The answer: selenium is antifungal and anti-mycotoxin. (Costantini, A., et al. Etiology and Prevention of Prostate Cancer: Hope at Last. Fungalbionics series. Johann Reiedrich Oberlin Verlag. Freiburg, Germany. 1998/1999. pp 320-323).
Beggs, W. Anti-Candida activity of the anti-cancer drug Tamoxifen. Research communications in chemical pathology and pharmacology. 80(1). April 1993.

Tamoxifen is often given as a follow up treatment to surgery for breast cancer—it is given to decrease the recurrence of the breast cancer. How does it work? Not only is it anti-estrogen, which is the conventional reason for prescribing it in this fashion, but it is also antifungal. It exerts marked antifungal action against Saccharomyces cerevisiae (brewer's/baker's yeast) and Candida albicans.

### C. Examples of Antifungal Prescriptive Medications as Well as Naturally-Occurring Antifungal and Anti-Mycotoxin Supplements to be Used Either Alone or in Conjunction with a Carbohydrate-Sparring Diet in the Treatment of a Bloodstream or Soft-Tissue Cancer

1. Fluconazole (Diflucan®, Apo-Fluconazole®) 200 mg in tablet or suspension form (10 mg/ml or 40 mg/ml) by mouth every other day for 30 days

2. Fluconazole (Diflucan®) 200-400 mg in tablet or suspension form (10 mg/ml or 40 mg/ml) by mouth or intravenously daily for 14 days

3. Fluconazole (Diflucan®) 200 mg in tablet or suspension form (10 mg/ml or 40 mg/ml) by mouth daily for three consecutive days, followed by 200 mg each Monday and Thursday thereafter for one month total

4. Fluconazole (Diflucan®) in any of the combinations listed in #1-3 above in combination and simultaneous with Nystatin (Mycostatin®) oral tablets, 500,000 units per tablet, 2 tablets twice a day for 30 days, or in combination with and simultaneous with any of the preparations of Nystatin listed below in #9-13.

5. Fluconazole (Diflucan®) 800 mg per day in tablet or suspension form (10 mg/ml or 40 mg/ml) intravenously for 7 days.

6. Fluconazole (Diflucan®) 200 mg by mouth in tablet or suspension form (10 mg/ml or 40 mg/ml) on day one, then 100 mg per day for the next 14 days.

7. Fluconazole (Diflucan®) 400 mg in tablet or suspension form (10 mg/ml or 40 mg/ml) by mouth daily for 3-12 months.

8. Fluconazole (Diflucan®) 400 mg in tablet or suspension form (10 mg/ml or 40 mg/ml) by mouth daily for 8 weeks.

9. Nystatin (Mycostatin®) oral tablets, 500,000 units per tablet, 2-3 tablets by mouth 2-4 times a day for 30 days, taken alone or in combination with a systemic antifungal agent.

10. Nystatin (Mycostatin®, Bio-statin®, Nystat-Rx®, Nystop®, Pedi-dri®, Nilstat®) oral suspension, 100,000 units per ml concentration, 2 cc by mouth twice a day for 14 days, taken alone or in combination with a systemic antifungal agent.

11. Nystatin (Mycostatin®, Bio-statin®, Nystat-Rx®, Nystop®, Pedi-dri®) oral suspension, 100,000 units per ml concentration, 1 cc in each side of the mouth four times a day for 10 days, taken alone or in combination with a systemic antifungal agent.

12. Nystatin (Mycostatin®, Bio-statin®, Nystat-Rx®, Nystop®, Pedi-dri®) oral suspension, 100,000 units per ml concentration, 5 cc by mouth, swished in the mouth and swallowed for 10 days, taken alone or in combination with a systemic antifungal agent.

13. Nystatin compounded powder, 500,000 units per ⅛ tsp, mixed in ½ cup of water and taken by mouth 4 times a day for 30 days, taken alone or in combination with a systemic antifungal agent.

14. Itraconazole (Sporanox®) in any of the following doses and/or regimens, alone or in combination with any of the Nystatin preparations listed in #9-13 above:
a. 100 mg capsule or oral solution (10 mg/ml concentration) by mouth daily for 30 days
b. 100 mg capsule or oral solution (10 mg/ml concentration) by mouth every other day for 30 days.
c. 200 mg in capsule form or 200 mg of the oral solution (10 mg/ml concentration) by mouth twice a day for one week of each month for three consecutive months.
d. Any of the above regimens (a-c) above preceded by:
   i. a loading dose of 200 mg intravenously twice a day for four consecutive doses, or
   ii. 200 mg, either in capsule or oral solution (10 mg/ml) form by mouth, three times a day for 3 consecutive days.
e. 200 mg intravenously twice a day for four consecutive days, followed by 200 mg intravenously, daily for 14 days.
f. 200 mg per day in capsule or oral solution (10 mg/ml concentration) form by mouth for 3 months.
g. 200 mg per day in capsule or oral solution (10 mg/ml concentration) form by mouth for 6 months.
h. 200 mg per day in capsule or oral solution (10 mg/ml concentration) form by mouth for 9 months
i. 300 mg by mouth in capsule or oral solution (10 mg/ml concentration) form, twice a day for three days, followed by 200 mg twice a day for 12 weeks.

15. Terbinafine (Lamisil®, Apo-Terbinafine®, Gen-Terbinafines®, Novo-Terbinafine®, PMS-Terbinafine®) in any of the following doses, alone or in combination and simultaneously with any of the nystatin regimens in #9-13 above:
a. 250 mg tablet by mouth daily for 6 weeks
b. 250 mg tablet by mouth daily for 12 weeks
c. 250 mg tablet by mouth, twice a day for 3 weeks
d. 250 mg tablet by mouth daily for 2-8 weeks.
e. 250-500 mg by mouth daily for up to 16 months.
f. For children:
   i. 67.5 mg by mouth per day for 2-8 weeks for children weighing under 20 kg
   ii. 125 mg by mouth per day for 2-8 weeks for children weighing from 20-40 kg
   iii. 250 mg by mouth per day for children weighing over 40 kg.
g. 250 mg tablet by mouth every other day for 30 days.

16. Ketoconazole (Nizoral®, Apo-ketoconazole®, Ketoderm®, Novo-ketoconazole®) in the following doses and/or regimens, alone or in combination and simultaneously with any of the nystatin regimens in #9-13 above:
a. 200-400 mg by mouth daily for 2 weeks
b. 200-400 mg by mouth daily for 30 days
c. 200-400 mg by mouth daily for 6 months.
d. For children over 2: 3.3-6.6 mg/kg/day for anywhere from 1 week up to 6 months.

17. Clotrimazole (Mycelex®, Canesten®) 10 mg oral troche dissolved on tongue and swallowed 5 times a day for 14 days.

18. Caspofungin Acetate (Cancidas®): 70 mg loading dose intravenously on day 1, followed by 50 mg intravenously daily until the clinical status of the patient improves; taken alone or in combination and simultaneously with any of the nystatin regimens in #9-13 above.

19. Voriconazole (Vfend®): for children over 12 and adults—6 mg/kg intravenously every 12 hours for 2 doses, followed by 4 mg/kg intravenously every 12 hours until the clinical status of the patient improves, at which time the oral form of the medication—400 mg every 12 hours—is used in place of the intravenous form; taken alone or in combination and simultaneously with any of the nystatin regimens in #9-13 above.

20. Amphotericin B (ABLC®, Amphotec®, AmBisome®, ABCD®, Amphocil®, Fungizone®) in the any of the following doses and regimens, alone or in combination and simultaneously with any of the nystatin regimens in #9-13 above:
a. 1 mg/kg/day intravenously for 14 days
b. 0.5 mg/kg/day intravenously to a total dose of over 1500 mg.
c. 0.5 mg/kg/day intravenously to a total dose of 5-7 mg/kg
d. 0.5 mg/kg/day intravenously until clinical improvement is noted
e. 0.5-1.0 mg/kg/day intravenously for 7 days
f. 1 cc (100 mg) of the oral suspension form by mouth 4 times a day for 14 days.

21. Flucytosine (Ancobon®): 100 mg/kg/day by mouth every 6 hours until clinical improvement is noted in the patient; alone or in combination and simultaneously with any of the nystatin regimens in #9-13 above

22. Griseofulvin (Fulvicin®, Fulvicin-U/F®, Grifulvin-V®, Gris-PEG®) in any of the following doses and/or regimens, alone or in combination and simultaneously with any of the nystatin regimens in #9-13 above:
a. 500-1000 mg per day of the microsized formula orally for ½ to 6 months
b. 330-375 mg/day of the ultramicrosized formula orally for ½ to 6 months
c. For children:
   i. 10-15 mg of the microsized formula/kg body weight/day for ½ to 6 months
   ii. 5.5-7.3 mg of the ultramicrosized formula/kg/day for ½ to 6 months

23. “Natural” Antifungals:
a. Grapefruit seed extract: Citricidal® 33% —15 drops mixed in water, taken orally twice a day
b. Olive leaf extract, 900 mg twice a day for 30 days or until clinical improvement is noted
c. Garlic 1,000 mg fresh extract three times a day until clinical improvement is noted.
d. Burdock root (Arctium lappa): 1,000 mg daily until clinical improvement is noted
e. Caprylic Acid: 1500 mg three times a day until clinical improvement is noted.
f. Pau d'arco (Tabebuia impetiginosa): 1000 mg by mouth, three times a day until clinical improvement is noted.
g. Undecylenic acid: 250 mg three times a day until clinical improvement is noted.
h. Selenium: 200 mcg per day by mouth as an adjunct to a carbohydrate-sparing diet and either natural or prescriptive antifungals.
i. Zinc picolinate or zinc citrate: 30 mg daily by mouth as an adjunct to a carbohydrate-sparing diet and either natural or prescriptive antifungals.
j. Iodine (in this case, the prescriptive form: Potassium Iodide (SSKI®, Iosat®, Pima®, Lugol's solution, KI): 5 drops three times a day by mouth, increasing to 40-50 drops 3 times a day and continuing for 3-6 months.
k. Vitamin C, 2,000 mg per day by mouth as an adjunct to a carbohydrate-sparing diet and either natural or prescriptive antifungals.
l. Vitamin E, 400 IU twice a day by mouth as an adjunct to a carbohydrate-sparing diet and either natural or prescriptive antifungals.
m. Vitamin D, 400 IU daily by mouth as an adjunct to a carbohydrate-sparing diet and either natural or prescriptive antifungals.
n. Broccoli sprouts (containing sulfurophane), 250 mg capsule three times a day as an adjunct to a carbohydrate-sparing diet and either natural or prescriptive antifungals.

### D. The Initial Phase Diet

| | Foods that are | Foods that |
|---|---|---|
| | ALLOWED | are EXCLUDED |
| Food Groups | in the diet: | from the diet: |
| 1. Sugar | None (1) | All sugars should be excluded |
| 2. Artificial or | Stevia, Stevia Plus | Aspartame, saccharin |
| herbal sweeteners | | |
| 3. Fruit | Green apples, berries, | Melons, bananas, |
| | avocados | bottled or frozen fruit |
| | grapefruit, lemons, limes; | juice; |
| | most | dried or sundried |
| | fresh, unblemished fruits | fruits (raisins, etc.) |
| | and | |
| | freshly squeezed fruit | |
| | juice | |
| 4. Meat | Fish, poultry, beef, etc. (2) | Breaded meats |
| 5. Eggs | Yes, all eggs are allowed | Egg substitutes |
| | | should be avoided |
| 6. Dairy Products | Yogurt (especially goat | All others, including |
| (3) | yogurt), | margarine |
| | cream cheese, | and any butter |
| | unsweetened | substitute |
| | whipping cream, sour cream | |
| | made with real cream, butter | |
| 7. Vegetables | Most fresh, unblemished | Potatoes, legumes |
| | vegetables and freshly- | (beans and peas) |
| | made vegetable juice (4) | |
| 8. Beverages | Bottled or filtered water, | Coffee and tea |
| | non- | (including decaf) |
| | fruity herb teas, fresh | Sodas (including diet sodas) |
| | lemonade or lime-ade sweetened | |
| | with Stevia | |
| 9. Grains | No grains are allowed | Pasta, rice, corn, wheat, |
| | on the IPD | quinoa, |
| | | amaranth, millet, buckwheat, |
| | | oats, barley |
| 10. Yeast products | No yeast products are | All are excluded, including |
| | allowed | bread, mushrooms, pastries, |
| | on the IPD | and alcoholic beverages |
| 11. Vinegars | Unpasteurized apple | Pickles, salad dressings (5), |
| | cider vinegar, | green olives, soy sauce. |
| | black olives not aged in | |
| | vinegar | |
| 12. Oils | Olive, grape seed, flax | Partially-hydrogenated |
| | seed, etc. | (“trans”) |
| | Use cold-pressed when | oils, corn and peanut oil |
| | available | |
| 13. Nuts | Raw nuts, including | Peanuts (along with ALL |
| | pecans, | peanut |
| | almonds, walnuts, | products) and pistachios |
| | cashews, | are excluded. |
| | pumpkin seeds, | |
| | sunflower seeds, | |
| | etc. | |

(1) Honey may occasionally and sparingly be used as a sweetener if needed.
(2) Meat and fish are better if not corn-fed. This means avoiding farm-raised fish and meat, even if they are “organic.” Grass-fed beef is ideal.
(3) Dairy products are better if from range-fed cattle and animals not injected with antibiotics, hormones, or steroids nor fed silo-stored grains. Good products: Brown Cow, Monarch Hills, Redwood Hills. Whipping cream is liquid, unsweetened heavy cream.
(4) Organically grown vegetables are preferable.
(5) Excluded because many of them are fermented products

### A Week of Phase I

### An Example of One Week on the Phase I Diet

Since this diet is so diametrically opposed to the Standard American Diet, many inquiries have come my way regarding what a week of the Phase I diet looks like. It is not meant to be followed verbatim and is rarely limited to just one week; rather it is merely to serve as an example. Please note the emphasis on water. You may refer to our recipe section for details on certain dishes.

| | |
|---|---|
| MONDAY | |
| Breakfast: | Fried eggs, uncured bacon, ½ grapefruit |
| Snack: | Almonds, water (always bottled or filtered) |
| Lunch: | Tuna with celery. Herbal tea. |
| Snack: | carrot sticks, water |
| Dinner: | Steak, steamed veggies, sparkling lime water |
| (optional)Dessert: | Plain yogurt with raspberries |
| TUESDAY | |
| Breakfast: | Omelet with onions, leeks, parsley, and chopped bacon |
| Snack: | celery sticks, water |
| Lunch: | Chicken salad with Phase I dressing |
| Snack: | cashews, water |
| Dinner: | Salmon fillets with lemon and butter, avocado salad |
| (optional)Dessert: | green apple |
| WEDNESDAY | |
| Breakfast: | Poached eggs, freshly squeezed carrot juice |
| Snack: | walnuts, water |
| Lunch: | broccoli chicken without rice, herbal tea |
| Snack: | grapefruit, water |
| Dinner: | Steak, spinach salad with lemon juice |
| | and olive oil dressing |
| (optional)Dessert: | plain yogurt with chopped pecans and fresh cranberries |
| THURDAY | |
| Breakfast: | scrambled eggs with breakfast steak |
| Snack: | green apple wedges, almonds, herbal tea |
| Lunch: | tuna salad with lettuce |
| Snack: | broccoli, cauliflower, water |
| Dinner: | halibut with sauteed vegetables |
| (optional)Dessert: | yogurt with fresh blueberries |
| FRIDAY | |
| Breakfast: | freshly squeezed carrot juice, hard boiled eggs |
| Snack: | celery sticks or green apple wedges with almond |
| | or cashew butter |
| Lunch: | beef patties, steamed and buttered asparagus |
| Snack: | sunflower seeds, water |
| Dinner: | Kaufmann's favorite meal (see recipes) |
| (optional)Dessert: | ½ grapefruit |
| SATURDAY | |
| Breakfast: | Omelet with green onions, bacon, spinach leaves |
| Snack: | carrot sticks |
| Lunch: | Cucumber salad with onions, tomatoes, |
| | black olives, olive oil |
| Snack: | pecans, yogurt with blackberries, water |
| Dinner: | Steak with steamed broccoli |
| (optional)Dessert: | sautéed green apples and cranberries with roasted |
| | pecans and whipping cream |
| SUNDAY | |
| Breakfast: | Freshly squeezed carrot juice, ½ grapefruit, |
| | poached eggs |
| Snack: | pumpkin seeds, water |
| Lunch: | salad with grilled tuna, herbal tea |
| Snack: | celery sticks, water |
| Dinner: | Stir-fried chicken, broccoli, snow peas, |
| | squash with butter |
| (optional)Dessert: | almonds, chamomile tea |

Although preferred embodiments of the invention have been illustrated in the accompanying Drawings and described in the foregoing Detailed Description, it will be understood that the invention is not limited to the embodiments disclosed but is capable of numerous rearrangements, modifications, and substitutions of parts and elements without departing from the spirit of the invention.

1. A method of treating a mammal having cancer comprising administering to said mammal a formulation in an amount, at a frequency, and for a duration effective to reduce or eliminate said cancer, said formulation comprising an anti-fungal agent.

2. The method of claim 1 wherein the anti-fungal agent comprises an anti-fungal agent selected from the group consisting of fluconazole, nystatin, itraconazole, terbinalfine, ketoconazole, coltrimazole, caspofungin, voriconazole, amphotericinB, flucytosine, and griseofulvin.

3. The method of claim 2 including the additional step of administering to said mammal a low carbohydrate diet.

4. The method according to claim 3 wherein the low carbohydrate diet excludes all sugars.

5. The method according to claim 3 wherein the low carbohydrate diet excludes all sugars, aspartain, and saccharin.

6. The method according to claim 3 wherein the low carbohydrate diet excludes all fruits except green apples, berries, avocadoes, grapefruit, lemons, and limes.

7. The method according to claim 3 wherein the low carbohydrate diet excludes all bread and meats.

8. The method according to claim 3 wherein the low carbohydrate diet excludes all dairy products except yogurt, cream cheese, unsweetened whipping cream, sour cream, and butter.

9. The method according to claim 3 wherein the low carbohydrate diet excludes potatoes, yams, and legumes.

10. The method according to claim 3 wherein the low carbohydrate diet excludes coffee, tea, sodas, and canned juice.

11. The method according to claim 3 wherein the low carbohydrate diet excludes all grains and all food products made from grains.

12. The method according to claim 3 wherein the low carbohydrate diet excludes yeast products, including bread, pastries, mushrooms, and alcoholic beverages.

13. The method according to claim 3 wherein the low carbohydrate diet excludes pickles, salad dressings, and green olives.

14. The method according to claim 3 wherein the low carbohydrate diet excludes partially-hydrogenated oils and peanut oil.

15. The method according to claim 3 wherein the low carbohydrate diet excludes peanuts, all products made from peanuts and pistachios.

16. The method according to claim 3 wherein the low carbohydrate diet excludes all sugars, aspartain, and saccharin, all fruits except green apples, berries, avocadoes, grapefruit, lemons, and limes, all bread and meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, and green olives, partially-hydrogenated oils, peanut oil, peanuts, all products made from peanuts, and pistachios.

17. The method of claim 1 wherein the anti-fungal agent comprises an anti-fungal agent selected from the group consisting of grapefruit seed extract, olive life extract, garlic, burdok, root, caprylic acid, pau d-arco, undecylenic acid, selenium, zinc picolinate, zinc citrate, iodine, vitamin C, vitamin E, vitamin D, and broccoli sprouts.

18. The method of claim 17 including the additional step of administering to said mammal a low carbohydrate diet.

19. The method according to claim 17 wherein the low carbohydrate diet excludes all sugars.

20. The method according to claim 17 wherein the low carbohydrate diet excludes all sugars, aspartain, and saccharin.

21. The method according to claim 17 wherein the low carbohydrate diet excludes all fruits except green apples, berries, avocadoes, grapefruit, lemons, and limes.

22. The method according to claim 17 wherein the low carbohydrate diet excludes all bread and meats.

23. The method according to claim 17 wherein the low carbohydrate diet excludes all dairy products except yogurt, cream cheese, unsweetened whipping cream, sour cream, and butter.

24. The method according to claim 17 wherein the low carbohydrate diet excludes potatoes, yams, and legumes.

25. The method according to claim 17 wherein the low carbohydrate diet excludes coffee, tea, sodas, and canned juice.

26. The method according to claim 17 wherein the low carbohydrate diet excludes all grains and all food products made from grains.

27. The method according to claim 17 wherein the low carbohydrate diet excludes yeast products, including bread, pastries, mushrooms, and alcoholic beverages.

28. The method according to claim 17 wherein the low carbohydrate diet excludes pickles, salad dressings, and green olives.

29. The method according to claim 17 wherein the low carbohydrate diet excludes partially-hydrogenated oils and peanut oil.

30. The method according to claim 17 wherein the low carbohydrate diet excludes peanuts, all products made from peanuts, all products made from peanuts, and pistachios.

31. The method according to claim 17 wherein the low carbohydrate diet excludes all sugars, aspartain, and saccharin, all fruits except green apples, berries, avocadoes, grapefruit, lemons, and limes, all bread and meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, and green olives, partially-hydrogenated oils and peanut oil, peanuts, all products made from peanuts and pistachios.

32. A method of treating a mammal having cancer indicating symptoms comprising administering to said mammal a formulation in an amount, at a frequency, and for a duration effective to reduce or eliminate said symptoms, said formulation comprising an anti-fungal agent.

33. The method of claim 32 wherein the anti-fungal agent comprises an anti-fungal agent selected from the group consisting of fluconazole, nystatin, itraconazole, terbinalfine, ketoconazole, coltrimazole, caspofungin, voriconazole, amphotericinB, flucytosine, and griseofulvin.

34. The method of claim 33 including the additional step of administering to said mammal a low carbohydrate diet.

35. The method according to claim 34 wherein the low carbohydrate diet excludes all sugars.

36. The method according to claim 34 wherein the low carbohydrate diet excludes all sugars, aspartain, and saccharin.

37. The method according to claim 34 wherein the low carbohydrate diet excludes all fruits except green apples, berries, avocadoes, grapefruit, lemons, and limes.

38. The method according to claim 34 wherein the low carbohydrate diet excludes all bread and meats.

39. The method according to claim 34 wherein the low carbohydrate diet excludes all dairy products except yogurt, cream cheese, unsweetened whipping cream, sour cream, and butter.

40. The method according to claim 34 wherein the low carbohydrate diet excludes potatoes, yams, and legumes.

41. The method according to claim 34 wherein the low carbohydrate diet excludes coffee, tea, sodas, and canned juice.

42. The method according to claim 34 wherein the low carbohydrate diet excludes all grains and all food products made from grains.

43. The method according to claim 34 wherein the low carbohydrate diet excludes yeast products, including bread, pastries, mushrooms, and alcoholic beverages.

44. The method according to claim 34 wherein the low carbohydrate diet excludes pickles, salad dressings, and green olives.

45. The method according to claim 34 wherein the low carbohydrate diet excludes partially-hydrogenated oils and peanut oil.

46. The method according to claim 34 wherein the low carbohydrate diet excludes peanuts, all products made from peanuts and pistachios.

47. The method according to claim 34 wherein the low carbohydrate diet excludes all sugars, aspartain, and saccharin, all fruits except green apples, berries, avocadoes, grapefruit, lemons, and limes, all bread and meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, and green olives, partially-hydrogenated oils, peanut oil, peanuts, all products made from peanuts, and pistachios.

48. The method of claim 32 wherein the anti-fungal agent comprises an anti-fungal agent selected from the group consisting of grapefruit seed extract, olive life extract, garlic, burdok, root, caprylic acid, pau d-arco, undecylenic acid, selenium, zinc picolinate, zinc citrate, iodine, vitamin C, vitamin E, vitamin D, and broccoli sprouts.

49. The method of claim 48 including the additional step of administering to said mammal a low carbohydrate diet.

50. The method according to claim 48 wherein the low carbohydrate diet excludes all sugars.

51. The method according to claim 48 wherein the low carbohydrate diet excludes all sugars, aspartain, and saccharin.

52. The method according to claim 48 wherein the low carbohydrate diet excludes all fruits except green apples, berries, avocadoes, grapefruit, lemons, and limes.

53. The method according to claim 48 wherein the low carbohydrate diet excludes all bread and meats.

54. The method according to claim 48 wherein the low carbohydrate diet excludes all dairy products except yogurt, cream cheese, unsweetened whipping cream, sour cream, and butter.

55. The method according to claim 48 wherein the low carbohydrate diet excludes potatoes, yams, and legumes.

56. The method according to claim 48 wherein the low carbohydrate diet excludes coffee, tea, sodas, and canned juice.

57. The method according to claim 48 wherein the low carbohydrate diet excludes all grains and all food products made from grains.

58. The method according to claim 48 wherein the low carbohydrate diet excludes yeast products, including bread, pastries, mushrooms, and alcoholic beverages.

59. The method according to claim 48 wherein the low carbohydrate diet excludes pickles, salad dressings, and green olives.

60. The method according to claim 48 wherein the low carbohydrate diet excludes partially-hydrogenated oils and peanut oil.

61. The method according to claim 48 wherein the low carbohydrate diet excludes peanuts, all products made from peanuts, all products made from peanuts, and pistachios.

62. The method according to claim 17 wherein the low carbohydrate diet excludes all sugars, aspartain, and saccharin, all fruits except green apples, berries, avocadoes, grapefruit, lemons, and limes, all bread and meats, all dairy products except yogurt, cream cheese, unsweetened whipping cream, sour cream, and butter, potatoes, yams, and legumes, coffee, tea, sodas, and canned juice, all grains and all food products made from grains, yeast products, including bread, pastries, mushrooms, and alcoholic beverages, pickles, salad dressings, and green olives, partially-hydrogenated oils and peanut oil, peanuts, all products made from peanuts and pistachios.
